(19) United States
(12) Patent Application Publication
(10) Pub. No.: US 2008/0312166 A1
(43) Pub. Date: Dec. 18, 2008

(54) Reversal core pharmaceutical system and method

(76) Inventors: Daryl John Lynn, Villa Ridge, MO (US); Tracy L. Amigo, Columbus, OH (US); Brian Lynn, Land O'Lake, FL (US); Lawrence A. Lynn, Columbus, OH (US)

Correspondence Address: The Sleep and Breathing Research Institute, Suite 10, 1275 Olentangy River Road, Columbus, OH, 43212, US

(21) Appl. No.: 12/157,874

(22) Filed: Jun. 13, 2008

Related U.S. Application Data
(60) Provisional application No. 60/934,766, filed on Jun. 15, 2007.

(51) Int. Cl. A61K 31/7048; A61K 31/404; A61K 31/5513; A61K 31/52; A61K 31/4188; A61P 25/00; A61K 31/497; A61K 31/4985; A61K 31/4178

(52) U.S. Cl. 514/27; 514/419; 514/221; 514/263.34; 514/252.11; 514/249; 514/397; 514/300

(57) ABSTRACT

A medication delivery system is disclosed to mitigate or reverse the pharmacologic effect of a medication beyond the desired therapeutic window. The system provides sequential delivery of medications including a first agent which induces a physiologic change, a second agent for reversing the physiologic change, and a medication release delaying agent for delaying the release of the second agent so that, upon receipt of the medication delivery system into the body the first agent is released before the second agent so that the physiologic effect of the first agent is mitigated by the second agent. The system can be a pill with the first agent comprising an outer medication and the second agent an inner medication with the medication release delaying agent intermediate the outer medication and the inner medication. In an example, the first agent can comprise a sleep aid and the second agent an arousal agent.

This application claims priority of Provisional Application 06/934,766, filed Jun. 15, 2007, the contents of this application is incorporated by reference as if completely disclosed herein.

### BACKGROUND AND SUMMARY OF THE INVENTION

The prolongation of therapeutic action of medications beyond the desirable time of the therapeutic action is a major problem. This occurs due to variable ages of the patients, and various functional states of organ systems either responsive to the medication or which excrete and/or metabolize the medication. Sensitivity to medications and elimination rates are also affected by a wide range of genetic factors which are generally not a priori known by the patient or prescriber. This is particularly true with sedatives, narcotics, and hypnotics but it also can apply to many other medications. In an example, there are many medications, both prescribed and over the counter, which are used to induce and aid in the perpetuation of sleep or sedation. Unfortunately the lingering affects of the product beyond the “desired therapeutic time interval” often inhibit the natural awakening process. The negative affect of the drowsiness in the morning can sometimes be extreme enough to outweigh the positive affects of even a mild hypnotic or sedative on sleep the night before. Many drugs claim to have less of a drowsy affect on people in the morning, but depending on the individual taking them this may or may not be true. Most sleeping aids commonly cause morning sleepiness in susceptible individuals. This type of extension of the primary therapeutic action beyond the desired therapeutic time interval has led to short acting medications but these are often less effective because they do not provide action throughout the desired therapeutic interval. Furthermore, in part due to varying rates of metabolism, excretion, and sensitivity among patient groups, medications which are short acting in some patients are often long acting in others, so that a more rapid reversal process is sometimes desired. In one example, in the prior art, the reversal of sedation or sleepiness is provided by the injection of naloxone or a benzodiazepine antagonist but such an injection adds expense, requires that the patient stay until this is performed, and is not applicable to a wide range of therapeutic environments (such as the home).

In one embodiment of the invention a medication delivery system comprises a first agent which induces at least one physiologic change or action (which includes one or more pharmacologically induced changes and/or actions), a second agent for reversing at least a portion of at least one physiologic change induced by the first agent, and a medication release delaying agent for delaying the release of the second agent, such that the release of the second agent occurs after the release of the first agent. The delaying agent can be configured to delay the release of the second agent by at least one hour, four hours, or six hours, after the release of the first agent. The physiologic change can comprise an adverse side effect of the first agent or the primary therapeutic effect of the first agent. The first agent can comprise a centrally or peripherally acting depressant as for example, a sedative, a sedating antihistamine, a hypnotic, a hormone, melatonin, and/or a benzodiazepine, to name a few. The second agent can comprise a centrally or peripherally acting stimulant as for example at least one of caffeine, Modafinil, a stimulating hormone, an amphetamine, and/or a medication which decreases GABA-mediated neurotransmission, to name a few.

One embodiment of the present invention comprises a medication delivery system for example, a tablet, a capsule, a capsule inside a capsule, a caplet, to name a few, configured for reversing or mitigating common side effects of medication and particularly for reversing the primary effect of a medication after a delay (for example after the primary effect is no longer desirable). An example of this embodiment is discussed below for sleeping aid related medications. While the discussion relates to sleeping aids, this is only an example and the methods, configurations, and functionalities are applicable to a wide range of medication classes and types. According to one aspect of this example, a first therapeutic agent such as a prolonged release of melatonin or another possible sleeping aid substance will last through the night gradually. Before waking the amounts being emitted will lessen. Just before the individual awakens the core of the pill will be reached and ingredients will be released to counter the effect of the sleeping aid in such a way as to negate the drowsiness side effect normally experienced the next morning. There are many other possible drugs and ingredients that may be used to produce the sleep aiding affect besides melatonin. The active sleep aiding ingredient can be layered along with a coating to prolong the breakdown of the pill. This will ensure that the gradual release of the sleeping aid will eventually lessen and end at the core of the pill at the desired point in time before the individual awakens counter acting the affects of the sleeping aid to produce a healthy natural arousal in the morning. The layers of the pill can alternate between the sleeping aid and a coating that will break down slowly prolonging the emission of sleeping aid through the night. As the pill breaks down the layers of sleeping aid will thin out towards the center of the pill therefore emitting less and less of the sleeping aid before the core is reached to wean the body off of the aid before reversal of affect. The “Reversal Core” will have one or more alerting agents, stimulants, and/or vitamins and/or minerals and/or plants such as Hoodia Gordonii, Ginseng, and/or Guarana to name a few, to bring about the negation of the affects of the sleeping aid. This will bring the individual to a state of normality with the natural level of energy preferred when waking. The alternating “Layered Release System” can also be used in the “Reversal Core” itself which could further the smoothness of the transition between the affects of the sleeping aid and the affects of the core meant to reverse the sleeping aids affects in preparation for waking.

One embodiment of the present invention, comprises a medication delivery system which comprises a first agent which induces a physiologic change, a second agent for reversing at least a portion of the physiologic change induced by the first agent, and a medication release delaying agent for delaying the release of the second agent, such that the release of the second agent occurs after the release of the first agent. According to one embodiment of the present invention a system and method is provided for a layering release system which can be configured to control intermittent release of a substance through a prolonged period of time. This layering release system can control frequency, volume, and time of release by using a spacer substance to separate the release times of the active substance. This system should not be limited to use with only sleeping aids and could be used to release any active substance with control of frequency, volume, and time of release. One embodiment comprises a medication delivery system comprising: a beneficial agent for inducing a beneficial change, the beneficial agent potentially inducing a known adverse side effect, a protecting agent which mitigates the adverse side effect, and a medication release delaying agent intermediate the beneficial agent and the protecting agent for delaying the release of the beneficial agent so that, upon receipt of the delivery system into the body, the protecting agent is released before the beneficial agent so that the adverse side effect of the beneficial agent can be more promptly mitigated by the protecting agent. Another embodiment comprises a medication delivery system comprising: a beneficial agent for inducing a beneficial change, the beneficial agent potentially inducing a delayed adverse side effect, a protecting agent which mitigates the delayed adverse side effect, and a medication release delaying agent intermediate the beneficial agent and the protecting agent for delaying the release of the protecting agent so that, upon receipt of the delivery system into the body, the protecting agent is released after the beneficial agent so that the delayed adverse side effect of the beneficial agent can be mitigated by the protecting agent.

According to the present invention a system and method is provided for a reversal core. This core at the center of a pill is comprised of a substance to counteract or negate the initial affects of the pill. This “Reversal Core” can also be layered using the layering release system before mentioned in order to control the process. This “Reversal Core” system could be used to reverse or counteract the affects of any active substance and is not limited to use with only sleeping aids.

Each agent within the product may be released at various intervals or may be released slowly from a layered release system, a matrix release system, or other controlled, sustained, or repeating release system. Some agents may be released immediately to bring about the onset of the desired action and may be followed by a delayed release of such agent to extend the action as well as a delayed release of the reversal agent. In an example, one embodiment comprises a medication on delivery system wherein the first component comprises diphenhydramine 25 mg to 50 mg or melatonin 1 to 3 mg as a sleep aid followed in later release by caffeine 50-100 mg as an arousal agent. One embodiment comprises a medication delivery system wherein the first component comprises methotrexate followed in later release by folic acid or a derivative or analog thereof. One embodiment comprises a medication delivery system wherein the first component comprises zolpidem tartrate 2.5 mg to 12.5 mg or temazepam 7.5 to 30 mg or eszopiclone 1 mg to 3 mg as a sleep aid followed in later release by caffeine 50-100 mg as an arousal agent. One embodiment comprises a medication delivery system wherein the first component comprises ondansetron or metoclopramide as an anti-nausea agent followed by etoposide or temozolomide as a chemotherapy agent later being released after protection is achieved. If nausea is expected to be of slow onset after a chemotherapy agent is released, the chemotherapy agent may be the first component. Given this teaching and disclosure, many other examples will be readily apparent to those skilled in the art. According to the present invention one method comprises the use of layers of CM-type hydroxyethylcellulose (HEC) in between layers of the sleep aid.

One purpose of the present invention is to provide by means of a release, which can be a prolonged release, a therapeutic agent followed by a release of a second agent to promptly counteract the pharmacologic effects of the therapeutic agent after the pharmacologic effects are no longer desired.

One purpose of the present invention is to provide controlled release of a therapeutic agent, such as a hypnotic enabling the controlled drop in dose of the sleeping aid until the “Reversal Core” is reached allowing the body to slowly be weaned off of the therapeutic agent before the affects are reversed (for example, prior to waking).

One purpose of the present invention to provide by means of systematic layers of a therapeutic agent such as a sleeping aid and layers of a substance to prolong breakdown of the pill to control the timing of when the core containing the reversal agent is reached.

One purpose of this present invention is to deliver melatonin or other ingredients to the body which would be beneficial to healthy sleep cycles as well as an antioxidant promoting a healthy heart and lower cholesterol followed by the delivery of an alerting agent.

One purpose of this present invention is to deliver melatonin or other ingredients to the body which would strengthen the natural circadian rhythm associated with healthy sleep followed by the delivery of an alerting agent.

One purpose of this present invention is to deliver melatonin or other ingredients to the body which comprise natural hormone (such as those secreted by the pineal gland) such that they are conducive to natural healthy sleep followed by the delivery of an alerting agent.

One purpose of this present invention is to deliver ingredients to the body which would be conducive to healthy sleep followed by the delivery of an alerting agent.

One purpose of this present invention is to induce healthy sleep followed by a healthy arousal the following morning.

### BRIEF DESCRIPTION OF THE DRAWINGS

These as well as other objects and advantages of this invention will be more completely understood and appreciated by careful study of the following more detailed description of the presently preferred exemplary embodiments of the invention taken in conjunction with the accompanying drawings, in which:

FIG. 1 is a cut-away view of a pill with a layered interior surrounding a central “Reversal Core.”

FIG. 2 is a cut-away view of a pill with a layered interior surrounding a central “Reversal Core” after the pill has been broken down for approximately six hours.

FIG. 3 is a side view of the “Reversal Core” after the exterior has been fully broken down leaving the core alone. This state is reached after approximately seven and a half hours.

### DESCRIPTION OF THE PRESENTLY PREFERRED EMBODIMENTS

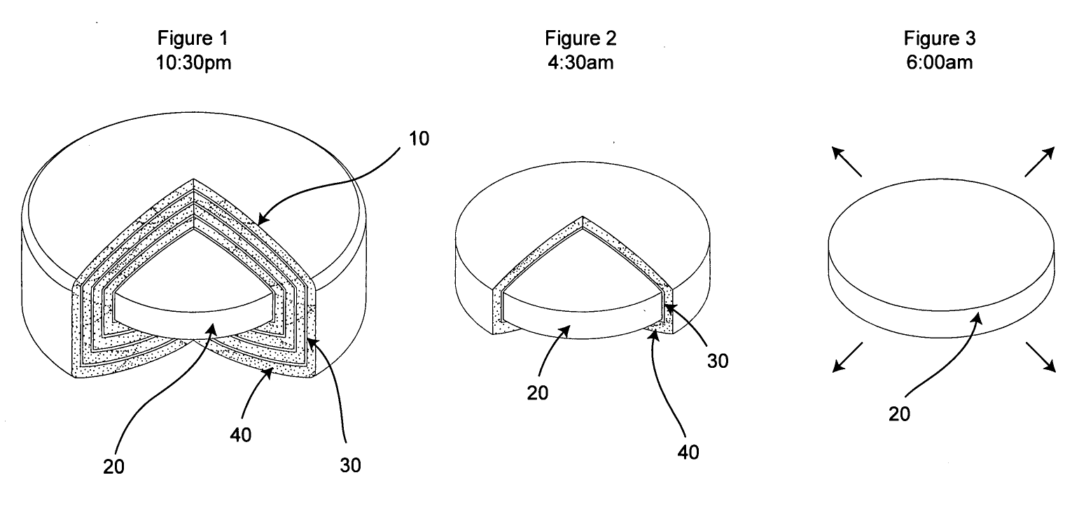

As shown in FIGS. 1, 2, and 3 one preferred embodiment of a sleeping aid pill with a “Layered Release System” and a “Reversal Core” is shown at three stages of functionality. The breakdown of the pill is an ongoing process and would have continual changes beyond the three stages that are given for example.

FIG. 1 shows the cut-away view of the first stage of the pill showing the interior of the pill as a layered system 10 that releases the sleeping aid in a controlled fashion through out the night. The release is controlled through the alternating layering of a coating 30 such as HEC to slow the pills breakdown and a layer of the sleeping aid substance 40. The layers of the sleeping aid substance 40 will thin and end before the “Reversal Core” 20 containing the alerting agent such as caffeine or Provigil™ is reached. This will promote a smooth transition from the effects of the sleeping aid into the effects of the “Reversal Core” 20 designed to work against the sleeping aid effects to negate them.

FIG. 2 shows the cut-away view of the second stage of the pill approximately six hours into the process. It shows the pill after being broken down with a thin layer of sleeping aid 40 remaining. It further shows a thicker layer of the substance used to slow down pill break down 30 to give a controlled period of time without sleeping aid before the “Reversal Core” 20 is reached to negate the sleeping aids effects before the individual awakens.

FIG. 3 shows the “Reversal Core” 20 being broken down and absorbed. It is possible to further add to the smooth transition between the effects of the sleeping aid and the “Reversal Core” 20 the same layering technique to the core for a more gradual effect from the core. This stage is reached at approximately seven to eight hours into the process although other time delays can be used.

In one embodiment the medication delivery system comprises a first outer agent which induces a physiologic change, a second inner agent for reversing the physiologic change, and a medication release delaying agent for delaying the release of the second so that, upon receipt of the medication into the body, the first agent is released before the second agent. The medication release delaying agent can be intermediate the outer agent and the inner agent.

One embodiment comprises a medication delivery system which comprises a beneficial agent for inducing a beneficial change, the beneficial agent potentially inducing a known adverse side effect, a protecting agent which mitigates the adverse side effect, and a medication release delaying agent intermediate the beneficial agent and the protecting agent for delaying the release of the beneficial agent so that, upon receipt of the delivery system into the body, the protecting agent is released before the beneficial agent so that the adverse side effect of the beneficial agent can be more promptly mitigated by the protecting agent. The release of the protecting agent for example can be delayed by 10-30 minutes or more in relation to the release of the beneficial agent so that the protecting agent has achieved sufficient concentration to promptly protect against the side effect of the beneficial agent upon the release of the beneficial agent.

One embodiment comprises a medication delivery system having a beneficial agent for inducing a beneficial change, the beneficial agent potentially inducing a delayed adverse side effect, a protecting agent which mitigates the delayed adverse side effect, and a medication release delaying agent intermediate the beneficial agent and the protecting agent for delaying the release of the protecting agent so that, upon receipt of the delivery system into the body, the protecting agent is released after the beneficial agent so that the delayed adverse side effect of the beneficial agent can be mitigated by the protecting agent.

One embodiment comprises a method for reversing prolonged effects of a medication which comprises, administering a medication delivery system having a first agent which induces a beneficial physiologic change, a second agent for reversing the beneficial physiologic change at a predetermined time when the beneficial change is no longer desired, and a medication release delaying agent for delaying the release of the second agent until the beneficial change is no longer desired. For example, the first agent can be a receptor agonist (such as an opioid, benzodiazepine, and/or beta agonist to name a few) and the second agent a receptor antagonist (such as an opioid antagonist, a benzodiazepine blocker, and/or a beta blocker to name a few. The first agent can be a receptor antagonist and the second agent a receptor agonist. The first agent can be a therapeutic agent (such as a chemotherapeutic agent) and the second agent a blocker of the action of the therapeutic agent for example an agent which chemically combines with a therapeutic agent to block its effect.

Although the presently preferred embodiments of this invention have been described, it will be obvious to those skilled in the art that various changes and modifications may be made therein without departing from the invention. In an example other medications in the outer layers with matching reversal core in the center can be provided wherein the reversal core counteracts the effects of the outer layers. Therefore, the claims are intended to include all such changes and modifications which may be made therein without departing from the invention. Therefore, the claims are intended to include all such changes and modifications that fall within the true spirit and scope of the invention.

What is claimed is:

1. A medication delivery system comprising: a first agent which induces a physiologic change, a second agent for reversing at least a portion of the physiologic change induced by the first agent, and a medication release delaying agent for delaying the release of the second agent, such that the release of the second agent occurs after the release of the first agent.

2. The medication delivery system of claim 1 wherein the delaying agent is configured to delay the release of the second agent by at least one hour after the release of the first agent.

3. The medication delivery system of claim 1 wherein the delaying agent is configured to delay the release of the second agent at least 4 hours after the release of the first agent.

4. The medication delivery system of claim 1 wherein the delaying agent is configured to delay the release of the second agent by at least 6 hours after the release of the first agent.

5. The medication delivery system of claim 1 wherein the physiologic change comprises an adverse side effect of the first agent.

6. The medication delivery system of claim 1 wherein the physiologic change comprises the primary therapeutic effect of the first agent.

7. The medication delivery system of claim 1 wherein the first component comprises a centrally or peripherally acting depressant.

8. The medication delivery system of claim 1 wherein the first component comprises a centrally or peripherally acting stimulant.

9. The medication delivery system of claim 1 wherein the first component comprises at least one of a sedative, hypnotic, a hormone, central nervous system depressant, melatonin, and benzodiazepine.

10. The medication delivery system of claim 1 wherein the second component comprises at least one of caffeine, Modafinil, a stimulating hormone, an amphetamine, a medication which decreases GABA-mediated neurotransmission, and central nervous system depressant.

11. A medication delivery system comprising: a first outer agent which induces a physiologic change, a second inner agent for reversing the physiologic change, and a medication release delaying agent for delaying the release of the second agent so that, upon receipt of the medication delivery system into the body, the first agent is released before the second agent.

12. The medication delivery system of claim 11 wherein the medication release delaying agent is intermediate the outer agent and the inner agent.

13. The medication delivery system of claim 11 wherein the first agent comprises a sleep aid and the second agent comprises an arousal agent.

14. The medication delivery system of claim 11 wherein the first agent comprises an antihistamine or melatonin as a sleep aid and the second agent comprises caffeine as an arousal agent.

15. The medication delivery system of claim 11 wherein the first agent comprises diphenhydramine 25 mg to 50 mg or melatonin 1 to 3 mg as a sleep aid and the second agent comprises caffeine 50-100 mg as an arousal agent.

16. The medication delivery system of claim 11 wherein the first agent comprises zolpidem tartrate 2.5 mg to 12.5 mg or temazepam 7.5 to 30 mg or eszopiclone 1 mg to 3 mg as a sleep aid and the second agent comprises caffeine 50-100 mg as an arousal agent

17. The medication delivery system of claim 11 wherein the first agent comprises methotrexate and the second agent comprises folic acid or a derivative or analog thereof.

18. A medication delivery system of claim 11 wherein the first agent comprises an anti-nausea agent and the second agent comprises a chemotherapy agent the second agent being released after protection against nausea is achieved by the first agent.

19. A medication delivery system of claim 11 wherein the first agent comprises ondansetron or metoclopramide as an anti-nausea agent and the second agent comprises etoposide or temozolomide as a chemotherapy agent the second agent being released after protection is achieved by the first agent.

20. A method for reversing prolonged effects of a medication comprising: administering a medication delivery system having a first agent which induces a physiologic change, a second agent for reversing the physiologic change, and a medication release delaying agent for delaying the release of the second agent, such that the release of the second agent occurs after the release of the first agent.
